(19) United States
(12) Patent Application Publication
(10) Pub. No.: US 2022/0412725 A1
(43) Pub. Date: Dec. 29, 2022

(54) Photogrammetric measurement of body dimensions using patterned garments

(71) Applicant: LIKE A GLOVE LTD., Petach Tikva (IL)

(72) Inventors: Simon Cooper, Ashdod (IL); Michael Slobodkin, Maple (CA)

(21) Appl. No.: 17/771,472

(22) PCT Filed: Mar. 19, 2020
(86) PCT No.: PCT/IB2020/052526

§ 371 Date: Apr. 24, 2022

Related U.S. Application Data
(60) Provisional application No. 62/937,265, filed on Nov. 19, 2019.
(60) Provisional application No. 62/939,730, filed on Nov. 25, 2019.

(51) Int. Cl. G01B 11/16; G06T 7/60; H04N 5/225; H04N 5/232; G06T 7/73; G01B 11/24; G06K 7/14

(52) U.S. Cl. CPC G01B 11/165; G06T 7/60; H04N 5/2254; H04N 5/2258; H04N 5/2256; H04N 5/23222; G06T 7/73; G01B 11/24; G06K 7/1417; G06T 2207/30244; G06T 2207/30196

(57) ABSTRACT

Measurement apparatus (20) includes a garment (24, 26, 140, 160) including an elastic fabric (27) having a predefined pattern (28) extending across a surface thereof and configured to be worn over a part of a body of a subject (22), such that the elastic fabric stretches across the part of the body. A camera (30, 80) is configured to capture images of the pattern while contacting and traversing across the surface of the fabric while the subject wears the garment. At least one processor (32, 36, 60) is configured to process the images captured by the camera at multiple locations on the surface of the fabric so as measure a local deformation of the pattern at the multiple locations due to stretching of the fabric, and to compute a dimension of the part of the body responsively to the measured deformation.

### CROSS-REFERENCE TO RELATED APPLICATION

This application claims the benefit of U.S. Provisional Patent Application 62/937,265, filed Nov. 19, 2019, and of U.S. Provisional Patent Application 62/939,730, filed Nov. 25, 2019, both of which are incorporated herein by reference.

### FIELD OF THE INVENTION

The present invention relates generally to measurement of body size and shape, and particularly to apparatus and methods for automating such measurements.

### BACKGROUND

Accurate measurement of the size and shape of a human body can be useful in a number of applications. Such measurements can be used, for instance, in identifying and purchasing articles of clothing that will fit a particular customer, as well as tracking changes in body proportions and shape for purposes of health and fitness monitoring.

As one example of this sort of application, PCT International Publication WO 2015/181661, whose disclosure is incorporated herein by reference, describes measurement apparatus, including an elastic fabric, configured as a garment to be worn over a part of a body of a human subject. One or more conductive fibers are integrated with the elastic fabric to as to stretch together with the elastic fabric when worn over the part of the body. A controller is coupled to measure an electrical property of the one or more conductive fibers in response to stretching of the elastic fabric, and to output an indication of a dimension of the part of the body based on the measured property.

Some systems extract body size measurements by processing images of the body. For example, PCT International Publication WO 2019/189846 describes a size measuring system having a size measuring instrument, which has, disposed on the surface thereof, a plurality of identifiable markers and which is attached to the body of a user when measuring the size of the body of the user. A measurement terminal measures the size of the body of the user by photographing the user having the size measuring instrument attached thereto.

As another example, U.S. Patent Application Publication 2017/0156430 describes a method for virtually selecting clothing, which is carried out on the basis of at least two photographs of the body of a subject dressed in an elastic template having reference markings. A computer processes the markings to produce a three-dimensional mathematical model of the body of the subject. Standard-shape graphical elements are applied, in a regular pattern, to an elastic covering which is worn on the body, and the relationship between the elements is used as a basis for forming a three-dimensional model of the body.

Similarly, U.S. Pat. No. 8,908,928 describes methods and systems for generating a size measurement of a body part of person for fitting a garment. The methods include providing photographic data that includes images of the body part and using feature extraction techniques to create a computer model of the body part.

### SUMMARY

Embodiments of the present invention that are described hereinbelow provide improved methods, systems and garments for use in measuring body dimensions.

There is therefore provided, in accordance with an embodiment of the invention, measurement apparatus, which includes a garment including an elastic fabric having a predefined pattern extending across a surface thereof and configured to be worn over a part of a body of a subject, such that the elastic fabric stretches across the part of the body. A camera is configured to capture images of the pattern while contacting and traversing across the surface of the fabric while the subject wears the garment. At least one processor is configured to process the images captured by the camera at multiple locations on the surface of the fabric so as measure a local deformation of the pattern at the multiple locations due to stretching of the fabric, and to compute a dimension of the part of the body responsively to the measured deformation.

In some embodiments, the predefined pattern includes multiple barcodes, which may include two-dimensional matrix barcodes. Additionally or alternatively, the predefined pattern includes at least one identification code, which may encode information identifying the garment. Further additionally or alternatively, the at least one identification code includes multiple graphical symbols, which are disposed at different, respective locations across the surface of the fabric and encode information identifying the respective locations.

In a disclosed embodiment, the predefined pattern includes one or more pattern colors that are distinguishable in the images from a background color of the fabric. Alternatively, the predefined pattern is formed in a pattern color that is indistinguishable to a human eye from a background color of the fabric.

In some embodiments, the predefined pattern is woven or knitted into the fabric and may have a patterned texture that is distinguishable in the images from a background texture of the fabric.

In a disclosed embodiment, the predefined pattern includes a code, which is disposed in proximity to a seam in the garment and is indicative of an extent of the fabric that is contained in the seam.

In some embodiments, the predefined pattern includes graphical symbols that are elongated in a vertical direction, relative to the body of the subject, while the elastic fabric is unstretched, and which expand horizontally when the subject wears the garment.

In some embodiments, the camera includes at least one image sensor and a housing, having a front end configured to contact the fabric as the camera traverses across the surface of the fabric. Objective optics are mounted in the housing and configured to image a plane at the front end of the housing onto the image sensor.

In one embodiment, the image sensor is contained in a mobile computing device, and the housing includes a fastener for attachment of the housing to the mobile computing device.

In another embodiment, the camera includes a wireless interface for transmitting data with respect to the images from the camera to the at least one processor.

In a disclosed embodiment, the at least one image sensor includes a plurality of image sensors configured to capture the images of the pattern along different, respective axes.

In some embodiments, the camera includes a window mounted at the front end of the housing so as to contact the surface of the fabric. In one embodiment, the window curves inward into the housing.

Additionally or alternatively, the camera includes one or more light sources disposed in the housing and configured to illuminate the fabric while the camera traverses across the surface. In a disclosed embodiment, the one or more light sources include a plurality of light sources, which are positioned in the housing so as to illuminate the fabric from different, respective angles. Additionally or alternatively, the one or more light sources are configured to direct flash illumination toward the fabric while the camera traverses across the surface.

In some embodiments, the at least one processor is configured to output an indication of the computed dimension to a user of the apparatus. In a disclosed embodiment, the predefined pattern includes an identification code, which identifies the garment, wherein the at least one processor is configured to process the images so as to read and validate the identification code, and to output the indication subject to finding the identification code to be valid. Additionally or alternatively, the at least one processor is configured to store values of the computed dimension, and to compare the computed dimension to the stored values, wherein the output is indicative of a change in the computed dimension relative to one or more of the stored values.

Alternatively, the at least one processor is configured to identify, responsively to the computed dimensions, one or more articles of clothing of a size suitable to be worn by the subject.

In a disclosed embodiment, the at least one processor is configured to track the locations on the surface of the fabric at which the camera captures the images, and to prompt a user of the apparatus, responsively to the tracked locations, to shift the camera to an area of the garment in which the images of the pattern have not yet been captured.

There is also provided, in accordance with an embodiment of the invention, a method for measurement, which includes providing a garment including an elastic fabric having a predefined pattern extending across a surface thereof and configured to be worn over a part of a body of a subject, such that the elastic fabric stretches across the part of the body. While the subject is wearing the garment, images of the pattern are captured using a camera, while the camera contacts and traverses across the surface of the fabric. The images captured by the camera are processed at multiple locations on the surface of the fabric so as measure a local deformation of the pattern at the multiple locations due to stretching of the fabric. A dimension of the part of the body is measured responsively to the measured deformation.

There is also provided, in accordance with an embodiment of the invention, a garment, including an elastic fabric, which is configured to be worn over a part of a body of a subject, such that the elastic fabric stretches across the part of the body, the fabric having a predefined pattern, which extends across a surface of the fabric and includes multiple graphical symbols, which are disposed at different, respective locations across the surface of the fabric and encode information identifying the respective locations.

There is further provided, in accordance with an embodiment of the invention, an imaging device, including a housing, having a front end configured to contact and slide across a surface that is to be imaged by the device. One or more light sources are contained in the housing and configured to illuminate the surface. At least one image sensor is contained in the housing, and objective optics mounted in the housing are configured to image a plane at the front end of the housing onto the image sensor.

In a disclosed embodiment, the device includes a processor configured to process images captured by the at least one image sensor. In one embodiment, the images include predefined symbols, which encode data and are disposed on the plane at the front end of the housing, wherein the processor is configured to decode the symbols in order to extract the data and to measure a deformation of the symbols in the images.

The present invention will be more fully understood from the following detailed description of the embodiments thereof, taken together with the drawings in which:

### BRIEF DESCRIPTION OF THE DRAWINGS

FIG. 1 is a schematic pictorial illustration of a system for measurement of body dimensions, in accordance with an embodiment of the invention;

FIG. 2 is a schematic sectional view of an imaging device, in accordance with an embodiment of the invention;

FIG. 3 is a schematic sectional view of an imaging device, in accordance with another embodiment of the invention;

FIG. 4A is a schematic pictorial illustration of an imaging module attached to a smartphone, in accordance with an embodiment of the invention;

FIG. 4B is a schematic frontal view of an image of a patterned garment captured using an imaging module, in accordance with an embodiment of the invention;

FIG. 5 is a schematic exploded view of an imaging module, in accordance with an embodiment of the invention;

FIG. 6 is a flow chart that schematically illustrates a method for measurement of body dimensions, in accordance with an embodiment of the invention;

FIG. 7 is a schematic representation of a user interface screen of a body measurement application, in accordance with an embodiment of the invention;

FIG. 8 is a schematic frontal view of a garment used in measurement of body dimensions, in accordance with an embodiment of the invention;

FIG. 9A is a schematic frontal view of a graphical symbol on a garment used in measurement of body dimensions, in accordance with an embodiment of the invention;

FIG. 9B is a schematic frontal view of the graphical symbol of FIG. 9A, as it appears when the garment is worn on the body of a subject;

FIG. 10 is a schematic frontal view of graphical symbols on a garment used in measurement of body dimensions, in accordance with another embodiment of the invention; and

FIG. 11 is a schematic frontal view of a garment used in measurement of body dimensions, in accordance with a further embodiment of the invention.

### DETAILED DESCRIPTION OF EMBODIMENTS

### Overview

There is a growing demand among consumers for accurate, convenient measurement of their body dimensions. For example, in on-line shopping applications, such measurements enable consumers to choose clothing whose size and style will fit them well, thus enhancing customer satisfaction and reducing the percentage of garments that are returned after purchase. As another example, people who have undertaken personal fitness programs have an interest in tracking the resulting changes in their body size and shape over time, such as reduction in their waist and hip circumferences, as well as increases in muscle size.

Embodiments of the present invention that are described herein address these needs by providing novel garments, cameras and software that facilitate such measurements. In the disclosed embodiments, a garment of this sort comprises an elastic fabric having a predefined pattern extending across its surface thereof. The garment fits snugly over a part of the body that is to be measured, such as a shirt, bra, or leggings, so that the elastic fabric stretches across the part of the body. Various sorts of suitable patterns are described hereinbelow. In some of these embodiments, the pattern comprises one or more identification codes, typically in the form of graphical symbols, which encode information identifying the garment and/or identifying the locations of the symbols on the garment. The patterns may be incorporated aesthetically and unobtrusively into general-purpose sportwear or other clothing, so that measurements can be carried out without requiring special-purpose garments dedicated for this purpose (although alternatively, garments dedicated for this purpose may be used).

A camera captures images of the pattern while contacting and traversing across the surface of the fabric while the subject wears the garment. In this manner, the images are captured at a fixed distance from the surface of the garment, and thus at a known ratio of image pixels to units of distance. This mode of operation of the camera obviates inaccuracies that commonly arise in non-contact measurement. The user simply slides the camera across the garment in order to capture images of the pattern at a sufficient number of locations. The local deformation of the pattern on the garment at each location is indicative of the degree of stretching of the fabric, and thus of the underlying body dimensions.

One or more processors process the images captured by the camera so as measure the local deformations. The measurement may be made locally, for example by an application running on a processor embedded in the camera or on the user's smartphone or other mobile computing device. Alternatively, image data may be transmitted over a network to a server for these purposes; or the processing may be performed in collaboration between one or more local devices and the server. In any case, the processor responsible combines the measurements of pattern deformation at different locations on the garment in order to compute one or more dimensions of the part of the body over which the garment is worn. The application then, for example, outputs an indication of the dimensions to the user in the form of body measurements and/or a 3D avatar, or in the form of a recommendation of an article of clothing to purchase, or a comparison of the current dimensions to earlier, stored values.

The image-based measurement techniques that are described herein may be combined with other measurement methods and devices that are known in the art, such as those described in the above-mentioned PCT International Publication WO 2015/181661, for purposes of enhancing accuracy and versatility of measurement.

### System Description

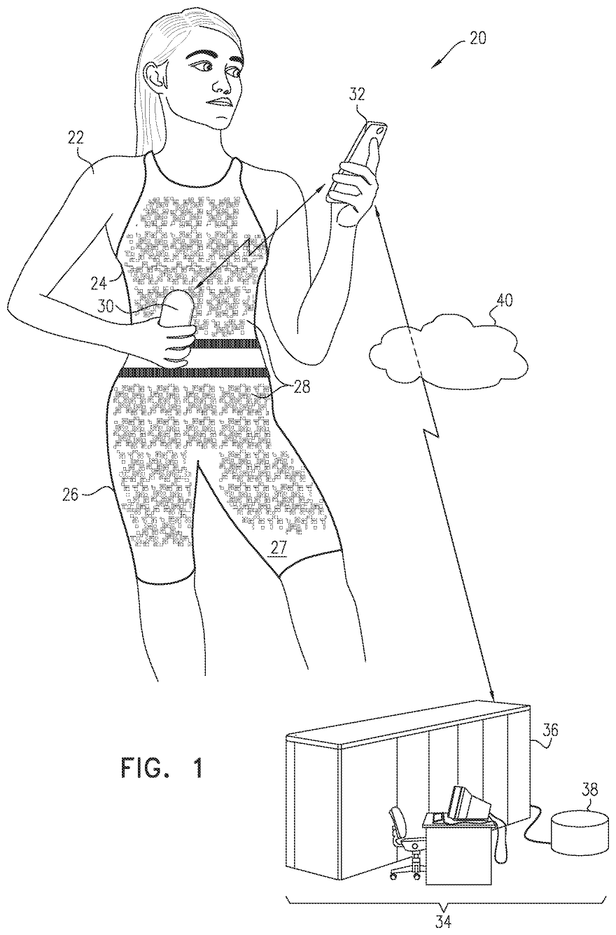

FIG. 1 is a schematic pictorial illustration of a system 20 for measurement of body dimensions, in accordance with an embodiment of the invention. A subject 22 wears garments 24, 26 comprising an elastic fabric 27 over her torso, abdomen and legs. Garments 24, 26 fit sufficiently snugly so that the fabric stretches across these parts of the subject's body. Any suitable elastic fabric can be used for this purpose, with any desired degree of stretch, from as little as 6% to as much as 300% or more, depending on design and application requirements.

A pattern 28 extends across the surface of fabric 27 and deforms as the shape of the body stretches the fabric. Details of this process, as well as patterns that may be used on garments in system 20, are described further hereinbelow. Although pattern 28 may appear in FIG. 1 to be uniformly periodic, and thus to repeat itself over the area of garments 24 and 26, in practice it can be advantageous that the pattern be non-uniform and possibly non-repeating. Specifically, in some embodiments, the pattern comprises graphical symbols that are disposed at different, respective locations across the surface of fabric 27 and encode information identifying the respective locations and possibly additional information, for example about garment type and model.

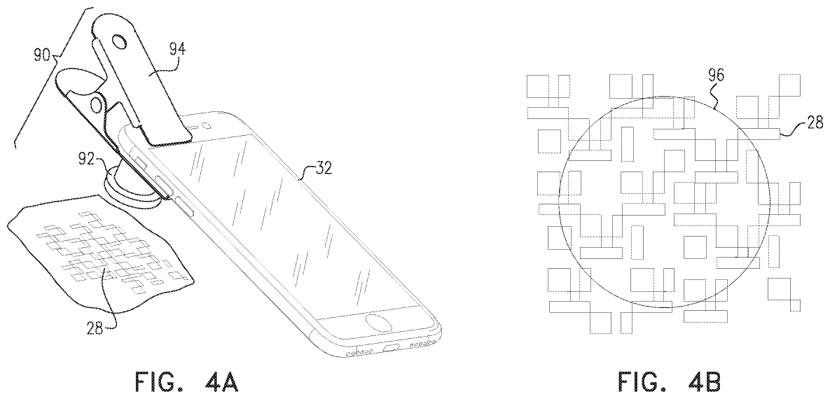

Subject 22 slides a camera 30 (likewise described hereinbelow) across the surface of fabric 27 of garment 24 and/or 26. Camera 30 captures images of pattern 28 and processes the images and/or transmits the images, for example via Bluetooth™ or another sort of wireless or wired connection, to an application run by the processor of a smartphone 32 or other computing device. (When the image processing is carried out by a microprocessor that is embedded in camera 30, the camera may transmit only the extracted data to smartphone 32, or it may transmit both extracted data and raw images.) As another alternative, the camera in smartphone 32 may be used to capture the images of the pattern (as described further hereinbelow with reference to FIGS. 4A/B).

In the pictured embodiment, smartphone 32 transmits image data to a server 34 for further processing. Server 34 comprises a processor 36 and a memory 38, and communicates with smartphone 32 over a network 40, such as the Internet. For example, smartphone 32 and/or camera 30 may extract certain parameters from the images captured by camera 30, and smartphone 32 may then transmit the parameters via network 40 to server 34. Additionally or alternatively, smartphone 32 may transmit raw images of pattern 28 to server 34 for processing. As yet another alternative, all of the processing described herein may be performed on camera 30 and/or smartphone 32 (or on another local computing device), using a suitable application running on the camera and/or smartphone. For example, a processor in camera 30 may process the raw images of pattern 28, detect information encoded in patterns 28 and the deformation of patterns 28, and send this processed information to smartphone 32, which in turn may optionally send this information to server 34. In any case, the images captured by camera 30 at multiple locations on the surface of fabric 27 are processed so as measure the local deformation of pattern 28 at each location due to stretching of the fabric, and then to compute, on the basis of the measured deformation, one or more dimensions of the parts of the body that are covered by garments 24 and/or 26.

System 20 is shown in FIG. 1, for the sake of concreteness and clarity, as comprising certain types of garments 24, 26, camera 30, and processing components. Alternatively, the principles of the present invention may be implemented using types of garments and cameras, as well as other processing components and system functionalities. All such alternative implementations are considered to be within the scope of the present invention.

### Camera Designs

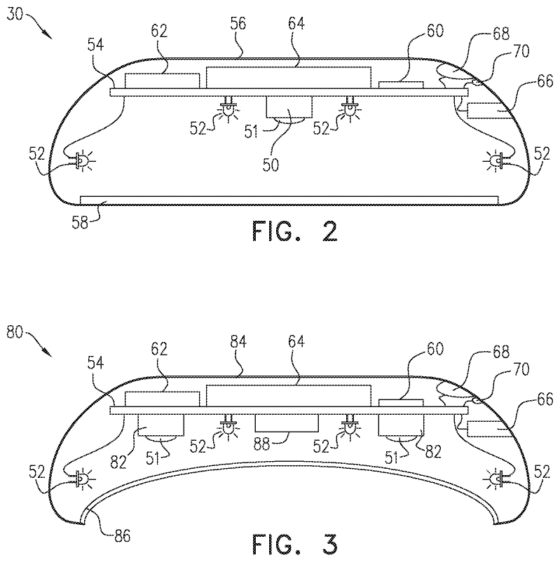

FIG. 2 is a schematic sectional view of camera 30, in accordance with an embodiment of the invention. Camera 30 is one example of an imaging device that can be used in system 20 and in other implementations of the present invention. A number of alternative imaging devices are shown in the figures that follow.

Camera 30 comprises an image sensor 50 with an objective optic, in the form of a lens 51, which focuses the surface of fabric 27 onto image sensor 50, or possibly multiple image sensors and optics. One or more light sources 52, such as suitable light-emitting diodes (LEDs), illuminate the fabric of garments 24, 26 while camera 30 traverses across the surface. In the pictured embodiment, light sources 52 are positioned in a housing 56 so as to illuminate the fabric from different, respective angles. The light sources may be operated in alternation, so that image sensor 50 captures images under different lighting conditions, which may be useful in enhancing the contrast of textured patterns. Alternatively or additionally, light sources 52 may be pulsed in order to direct flash illumination toward fabric 27 while camera 30 traverses across the surface. Operating in flash mode can be useful in reducing motion blur and other artifacts in the captured images. Alternatively or additional, image sensor 50 may operate in a shuttered mode.

Image sensor 50 and light sources 52 are mounted on or otherwise connected to a printed circuit board 54 inside a housing 56. The front end of housing 56 (i.e., the lower side in the view shown in FIG. 2) is configured to contact and slide across the surface that is to be imaged by camera 30. In other words, lens 51 images the plane at the front end of the housing onto image sensor 50. As the dimensions of housing 56 and the optical properties of lens 51 and camera 50 are known, the focus and magnification of fabric 27 onto image sensor 50 are also known and well controlled. Therefore, the ratio of the number of pixels per unit of distance is also known. To improve the focal control further, a transparent window 58 is mounted at the front end of housing 56 and presses against fabric 27, thus flattening fabric 27 as housing 56 slides across it. Window 58 may be rigid or slightly flexible. Alternatively, the front end of housing 56 may be open, without a window.

As camera 30 slides across fabric 27, it is important that housing 56 be pressed firmly against the fabric in order to ensure good image quality and accurate control of image magnification (i.e., the ratio of the number of pixels per unit of distance). For this purpose, light sources 52 may be operated selectively in order to assess the quality of contact between the front end of housing 56 and fabric 27 of garments 24 and 26. Specifically, when housing 56 is pressed firmly against the fabric, relatively little stray light should be able to reach image sensor 50. To verify that this is the case, image sensor 50 can be operated to capture an image with light sources 52 turned off. When the average luminance in this image is above a certainly limit, system 20 may issue an alert to the user, for example in the form of a visual or vocal notification by smartphone 32, indicating that the user should press camera 30 more firmly against the garment. Alternatively or additionally, a test light source (not shown) may be mounted on the outside of housing 56 and used to verify that light from the test light source does not reach image sensor 50 during measurements.

In the present embodiment, camera 30 also includes an embedded microcontroller 60 and a communication interface 62, such as a Bluetooth or Wi-Fi interface chip for wireless communications, or possible a wired interface. Microcontroller 60 (or a suitable microprocessor) controls the operation of image sensor 50 and light sources 52. The microcontroller also receives image data from image sensor 50, applies certain processing functions to the image data, and then transmits image frames and/or processed image data with respect to patterns 28 via interface 62 to smartphone 32 or to another processor. (For example, microcontroller 60 may detect and decode the patterns 28, estimate their deformation, and then transmit the resulting data to smartphone 32.) A battery 64 provides electrical power to the components of camera 30. An electrical receptacle 66, such as a Universal Serial Bus (USB) receptacle, may be provided in case 56 in order to receive an external cable for purposes of recharging battery 64, as well as data transfer and software updates. Camera 30 may also have a simple user interface, for example comprising an on/off button 68 and one or more indicator LEDs 70.

Camera 30 may optionally comprise other components, such as an inertial sensing unit, including a magnetometer, gyroscope, and/or accelerometer (not shown in this figure), which can be used to provide an indication of the orientation and possibly the location (relative to other images) at which camera 30 captured each successive image. Camera 30 may transmit this indication to smartphone 32 together with the image data and/or together parameters extracted from patterns 28 by microcontroller 60. The orientation information, and possibly the locations, of the images captured by camera 30 can be useful in estimating the body shape.

FIG. 3 is a schematic sectional view of a camera 80, in accordance with another embodiment of the invention. Camera 80 may be used in place of camera 30 in system 20, and includes many similar components, which are indicated by the same numbers in FIG. 3 and in FIG. 2. Only the points of difference between the embodiments will be discussed here.

Camera 80 comprises two image sensors 82, which capture respective images of pattern 28 along different, respective axes. Alternatively, camera 80 may comprise three or more image sensors. In this case, for example, each objective lens 51 may have a higher magnification, so that camera 80 will capture images with enhanced resolution. The use of multiple image sensors may also enable a thinner design of camera 80. Additionally or alternatively, the images captured by the two image sensors 82 may be combined in order to provide a stereoscopic view of the texture of pattern 28, for example, to assist in estimating the curve of the textile and thus better reconstruct the body shape. An inertial sensing unit 88 in camera 80 measures and outputs orientation data with respect to the captured images, as explained above.

Camera 80 comprises a housing 84 having a curved window 86 at its front end. Window 86 curves inward into the housing and thus allows the curves of the body of subject 22 to take their natural shape and be captured in the stereoscopic images taken by image sensors 82. Alternatively, the front end of housing 84 may be open, without a window at all.

Reference is now made to FIGS. 4A and 4B, which schematically illustrate an imaging module 90 attached to smartphone 32, in accordance with a further embodiment of the invention. FIG. 4A is a pictorial view, while FIG. 4B is a frontal view of an image 96 captured using module 90.

In this embodiment, the image sensor used to capture images of fabric 27 of garments 24 and 26 is the rear image sensor that is built into smartphone 32. A housing 92 contains objective optics, which focus an image of pattern 28 onto the image sensor when the front end of the housing is in contact with the fabric. A fastener, such as a clip 94, attaches housing 92 to smartphone 32 in alignment with the image sensor. Alternatively, any other suitable type of fastener may be used for this purpose.

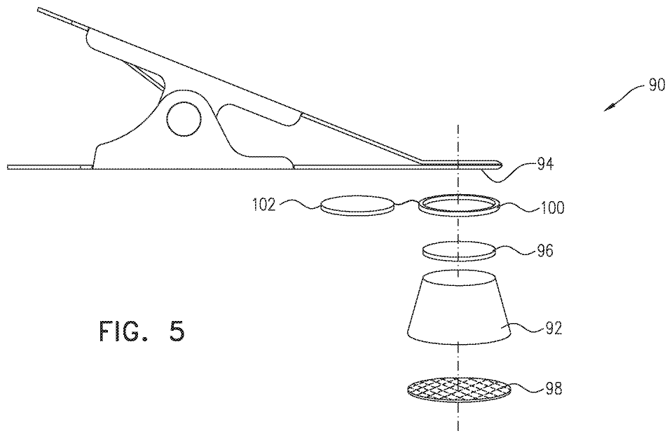

FIG. 5 is a schematic exploded view of imaging module 90, in accordance with an embodiment of the invention. Housing 92 contains an objective lens 96 and has a window 98 at its front end, for contacting and sliding over fabric 27 of garments 24 and 26. In this embodiment, window 98 includes a reticle grid on its surface to assist in calibrating the magnification of the images captured by the image sensor in smartphone 32; but alternatively, window 98 could have other sorts of grid patterns or no pattern at all. Optionally, housing 92 also contains a light source in the form of a ring light 100, which is powered by a battery 102 contained in or on clip 94. Alternatively, housing 92 may be semitransparent so to allow enough light to pass into the housing to illuminate pattern 28, so that a dedicated light source is not needed.

### Methods of Operation

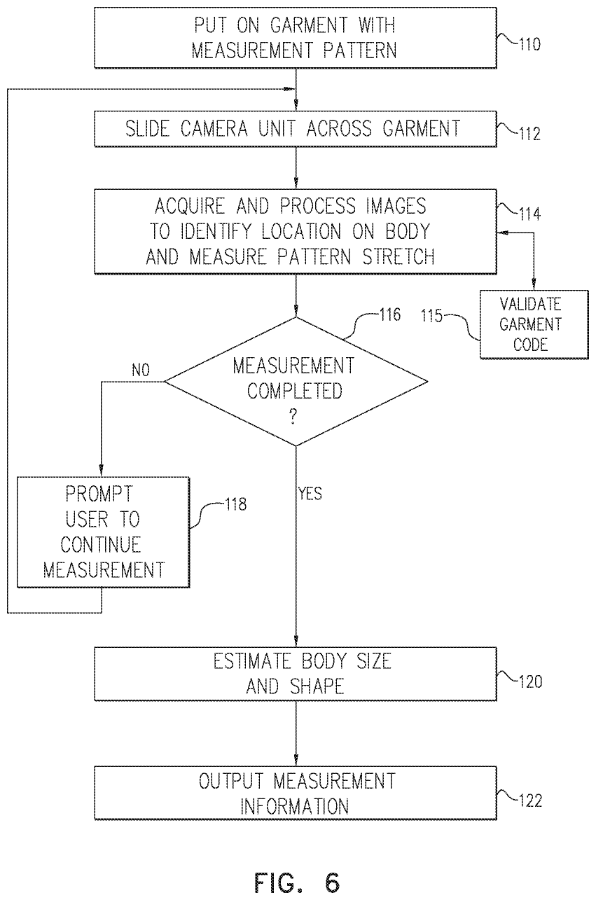

FIG. 6 is a flow chart that schematically illustrates a method for measurement of body dimensions, in accordance with an embodiment of the invention. The method is described here, for the sake of convenience and clarity, with reference to the elements of system 20, as shown in FIGS. 1 and 2. Alternatively, the method may be carried out using the cameras shown in FIG. 3 or FIG. 4A, or using any other suitable sorts of cameras and garments that satisfy the criteria set forth herein. The term “user” in the description below may refer either to subject 22 herself or to another user assisting the subject in the measurement process. Although FIG. 1 and the present language refer to a female subject, the principles of the present embodiments may equally apply to male subjects.

To initiate the present method, subject 22 puts on a garment with a measurement pattern, such as garment 24, at a dressing step 110. The user (i.e., the subject herself or an assisting user) opens the measurement application on smartphone 32 and actuates camera 30, for example by pressing button 68. The user then slides camera 30 across the surface of garment 24, at an image capture step 112. It can be advantageous that subject 22 stand still at this step, for example, by pressing her back against a wall while scanning the front part of garment 24.

Camera 30 automatically captures a series of image frames, each showing a part of pattern 28. Camera 30 may then transmit the image data to smartphone 32. Alternatively, camera 30 may process the images by itself, using software running on microcontroller 60, in which case it may be unnecessary to transmit the images to smartphone 32. (Alternatively, as explained above with reference to the embodiment of FIG. 4A, the camera in smartphone 32 may be used to capture the images, in which case such transmission is not needed.) Microcontroller 60 may also sense and record the orientation at which camera 30 captured each image frame, for example using an inertial sensor, such as a magnetometer and/or gyroscope in sensor 88 (FIG. 3), as described above.

A suitable processor processes the images captured at step 112 in order to identify the location on the subject's body at which each image was acquired and measure the pattern deformation in the image, at an image processing step 114. In the description that follows, it will be assumed that step 114 is carried out by microcontroller 60, since this approach is advantageous in reducing the volume of data that must be transmitted from camera to smartphone 32. Alternatively, however, step 114 may be carried out in whole or in part by the processor in smartphone 32 and/or by server 34. Furthermore, although step 114 is shown in FIG. 6 as following step 112, in practice these steps may be carried out concurrently, with microcontroller 60 or smartphone 32 processing a certain image at the same time as camera 30 acquires a subsequent image.

In the course of step 114, microcontroller 60 extracts the location at which each image was captured. The location can be identified on the basis of the unique local pattern that appears in the image, such as a graphical symbol that is indicative of the location. For example, in some embodiments, pattern 28 comprises barcodes, such as QR codes, which encode values that are indicative of the location. In this case, microcontroller 60 decodes the QR code and sends the resulting numerical value to smartphone 32 together with other image parameters. The application running on smartphone 32 converts the decoded value to a location on garment 24, either based on data stored on smartphone 32 or by submitting a query to server 34.

Alternatively or additionally, the processor may use the output of an inertial sensor in camera 30 in computing the position coordinates of camera 30 at which each image was captured. This coordinate information can be used in reconstructing the 3D shape of subject 22, based on the position in space of each image and its relation to other images.

Microcontroller 60 also identifies features in the pattern in each image, such as edges and corners, and then computes the distances between these features in order to measure the deformation of the pattern due to stretching of fabric 27. For example, microcontroller 60 may ascertain that a certain pair of features are now 8 mm apart, whereas the baseline distance between these feature, before garment 24 was stretched over the user's body, was only 4 mm. Alternatively, as noted earlier, at least some of these processing functions may be carried out by smartphone 32 and/or server 34.

Before proceeding with the measurements and further interaction with the user, microcontroller 60 may read and decode one or more symbols in pattern 28 on the garment, and may transmit the decoded values to smartphone 32 in order to verify that the garment is authentic, at a validation step 115. For example, it may be important to ensure that the garment was produced by an authorized manufacturer in order to prevent use of poor-quality imitation products that may yield inaccurate measurements. Furthermore, the user or the garment manufacturer may pay a fee, such as a subscription fee, for the measurement services provided by server 34 and/or by the application running on smartphone 32, in which case authentication ensures that the subscription is in order.

To enable such validation and authentication, pattern 28 on garment 24 includes, in some embodiments, an identification code at a predefined location, and the user may be prompted to slide camera 30 over this location at some stage in step 112. Alternatively, all of the symbols in pattern 28 may encode the identification code, along with location information. The processor in smartphone 32 then validates the identification code, for example by checking the value of the code in memory 38 of server 34, before proceeding with further measurements in step 114. The identification code may indicate the type of garment (leggings, shirt, etc.), as well as the style and version of the garment and the manufacturing batch, and it may also be used as a key in identifying the respective locations of the specific symbols making up the pattern on a given garment. Server 34 may look up this location information in memory 38 and use the information in processing image data transmitted by smartphone 32, for example, or it may return the location information to the smartphone for use by the measurement application. The identification code may be the same for all garments of a given type, or it may be varied from one manufacturing batch to the next, or even from garment to garment.

As a further alternative, for example, at the time of purchase, the salesperson may scan an identification code of the garment, transmit the information to server 34, and then receive and print a code on the receipt. The user scans this code using smartphone 32 while running the measurement application, and will then be able to scan and make measurements while wearing this specific garment. Attempts to use the measurement application on imitation garments, without the appropriate authorization code, will then fail.

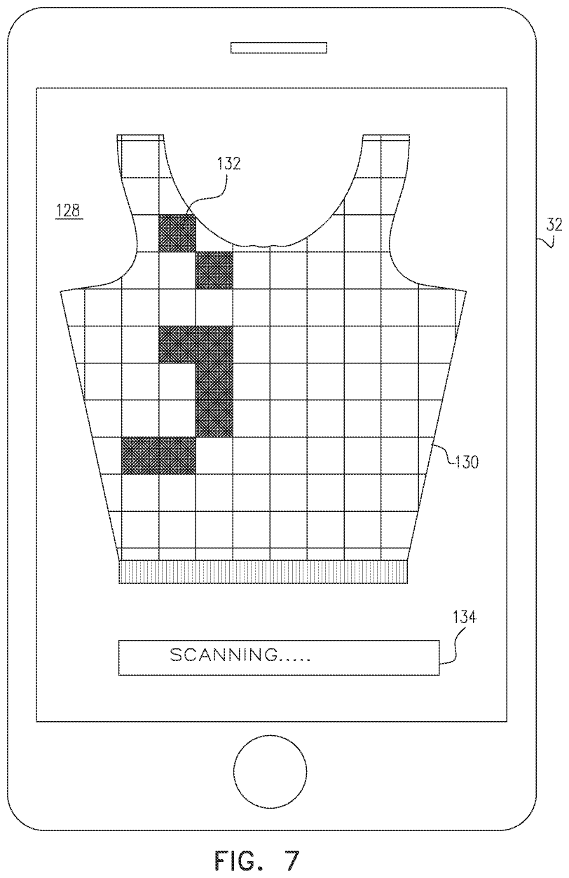

Assuming authentication (if necessary) is completed successfully at step 115, microcontroller 60 continues acquiring images and measuring pattern stretch at step 114, as the user continues sliding camera 30 across the garment. The processor in smartphone 32 checks the locations of the acquired parts of the pattern at a completion checking step 116. If there are still significant parts of garment 24 that have not been scanned, the processor prompts the user to continue the measurements, at a user prompting step 118. For example, the processor may issue a message via the user interface of smartphone 32 indicating the area of garment 24 that have not yet been scanned. (A user interface screen for this purpose is shown in FIG. 7.)

When the processor in smartphone 32 finds at step 116 that the measurements have been completed, it proceeds to compute one or more dimensions of the part of the body of subject 22 that is covered by garment 24, at a dimension computation step 120. More generally, the measurements that the processor has made of the local deformation of the pattern enable it to estimate and model the entire size and shape of this part of the subject's body. Alternatively, the user may decide to end the scan, whereupon the dimensions are computed on the basis of the measurements that have been made.

As another alternative, the processor may compute and dimensions of the body of subject 22 in parallel with step 114, based on the cumulative measurements that have been made at each point in the process. For example, the user may first run camera 30 down along the side of the body, giving measurements of deformation that will enable the processor to estimate the circumference of the body at each of a series of longitudinal coordinates. The processor may display these estimates and prompt the user to capture images at additional locations in order to fill in details of the body shape.

When step 120 has been completed, the processor in smartphone 32 outputs an indication of the computed dimension or dimensions to the user, at an output step 122. As noted earlier, the application running on smartphone 32 may first validate the identification code of garment 24 at step 115 before providing this output. The output at step 122 may present the body shape and size information in various forms, for example by showing a 3D avatar of the body, by presenting measurements of specific body parts, and/or by comparing values from different measuring sessions.

Specifically, when smartphone 32 or server 34 stores past values of the computed dimensions, the processor can compare the value of the dimension measured at step 120 to the stored values, and then provide the user with an indication of the change of the current dimension relative to the stored values. For example, the processor may display a trend illustrating the decrease is waist and hip size or increase in muscle size over the course of a program of exercise and weight loss. As another example, the changes in body dimensions can be reported to and used by a medical caregiver or physiotherapist for diagnosis and follow-up of the subject's medical and physiological condition.

Additionally or alternatively, the processor may use the computed dimensions in identifying one or more articles of clothing of a size suitable to be worn by subject 22, for example via an Internet shopping site. Methods for on-line shopping that can be applied using dedicated measurement garments, such as garments 24 and 26, are described, for example, in U.S. Pat. No. 9,858,611, whose disclosure is incorporated herein by reference.

FIG. 7 is a schematic representation of a user interface screen 128 of a body measurement application running on smartphone 32, in accordance with an embodiment of the invention. Screen 128 may be displayed at step 118 and/or step 114 in FIG. 6 in order to track the locations on the surface of fabric 27 at which camera 30 has captured images, and to prompt the user on this basis to shift the camera to areas of garment 24 in which images of pattern 28 have not yet been captured. In this example, screen 128 presents a graphical representation 130 of the garment, with areas 132 marked to indicate whether or not they have been scanned by the camera. A completion bar 134 may give an indication of the fraction of the area that has been covered.

### Garment Designs

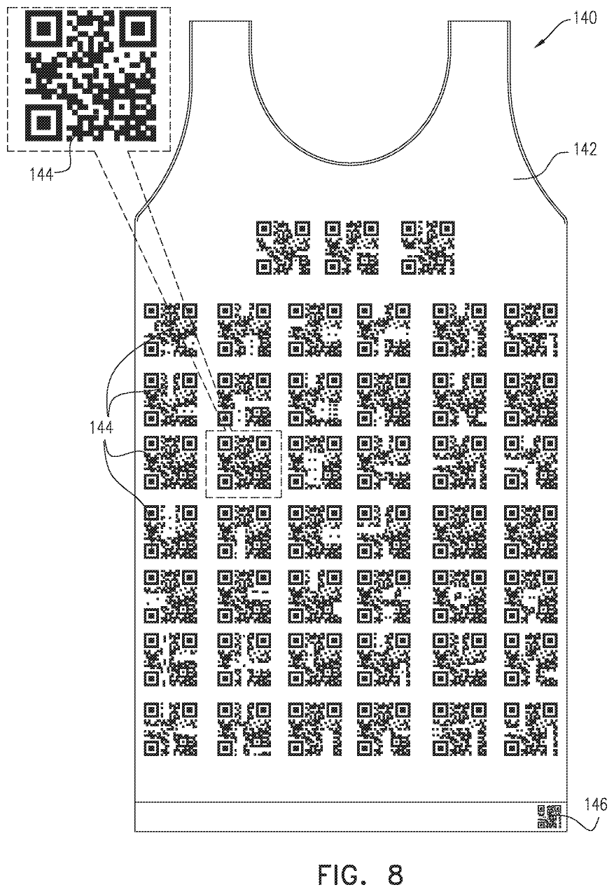

FIG. 8 is a schematic frontal view of a garment 140 used in measurement of body dimensions, in accordance with an embodiment of the invention. Garment 140 may be used in place of garment 24 in system 20 (FIG. 1), for example.

Garment 140 comprises an elastic fabric 142, which is sized so as to stretch of the subject's torso, and which is covered by a pattern of multiple graphical symbols in the form of barcodes 144. In this case, barcodes 144 comprise two-dimensional matrix barcodes, such as QR-codes, which are a convenient choice, because they are designed to be machine-readable, can encode a large range of numerical values, and include standardized error correction coding and registration marks. As noted earlier, barcodes 144 may be of same type but differ from one another across fabric 142 in terms of the data that they encode, and thus may encode information identifying their respective locations. In addition, at least one symbol 146 encodes information identifying garment 140, as explained above. Alternatively or additionally, the information identifying garment 140 may be encoded in some or all of barcodes 144, together with their respective locations. For ease and precision of image capture and processing, barcodes 144 may be sized so that they are slightly smaller than the field of view of camera 30; but alternatively, larger or smaller symbols may be used.

Barcodes 144 (or other symbols) may be incorporated into garment 140 using any suitable manufacturing technique that is known in the art. For example, the symbols may be printed or engraved onto the surface of fabric 142. Alternatively, the symbols may be woven or knitted into the fabric. For example, the pattern of the symbols may be knitted using yarns of different colors, or using yarns of different types, such as nylon and cationic polyester, which will then take on different colors when dyed.

Alternatively or additionally, when the pattern is woven or knitted, the pattern of the symbol may be embodied in a patterned texture, which is distinguishable in the images captured by camera 30 from the background texture of the fabric (which is typically smooth). The pattern may thus be woven or knitted, if desired, in the same color as the remaining fabric. The different illumination angles of light sources 52 (FIGS. 2 and 3) can be useful in highlighting the patterned texture.

Alternatively or additionally, the pattern may be formed on fabric 142 using any suitable color or combination of colors that will be distinguishable in the images captured by camera 30 from the background color of the fabric. The different colors may or may not be distinguishable to the human eye. For example, the colors of the pattern may be distinguishable, if desired, only under infrared or ultraviolet light, which is emitted by light sources 52.

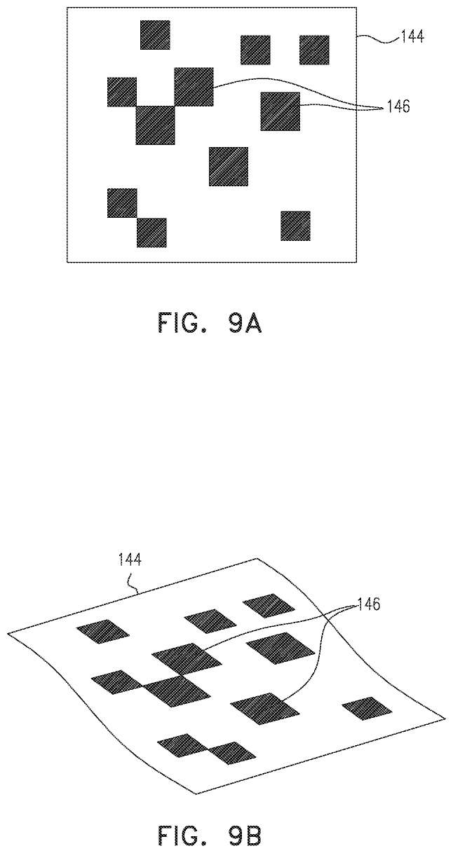

FIGS. 9A and 9B are schematic frontal views of a graphical symbol 144 on a garment used in measurement of body dimensions, in accordance with an embodiment of the invention. FIG. 9A shows symbol 144 as it appears on the garment as manufactured, while FIG. 9B shows symbol 144 as it appears when the garment is worn on the body of a subject. Symbol 144 comprises an array of graphical elements 146, whose locations within the symbol encode a numerical data value, such as a value indicating the location of this symbol on the garment.

Before the subject wears the garment, the features of graphical elements 146, such as the edges and corners, are separated by certain predefined distances, as illustrated in FIG. 9A. When the subject wears the garment, however, symbol 144 is deformed, as shown in FIG. 9B. The deformation is expressed in changes of the distances between graphical elements 146, as well as changes in angles and orientation (which may also result from rotation of camera 30). To identify and decode symbol 144, the processor (for example in camera 30, smartphone 32 or server 34) first applies image rectification and recognition algorithms, as are known in the art, in order to warp and align the deformed image of the symbol, as shown in FIG. 9B, with the baseline image shown in FIG. 9A, and to read the numerical data value encoded by the symbol.

The processor compares the distances between the deformed features of graphical elements 146 in the image (FIG. 9B) to those in the baseline image (FIG. 9A), and thus measures the deformation of symbol 144. Typically, as illustrated in this example, the deformation is a vector, with both horizontal and vertical components. Alternately or additionally, the deformation may be expressed in terms of curve and direction components. In practice, however, human bodies tend to vary much more in their girth than in their height, meaning that the horizontal deformation of the pattern due to stretching of the garment will typically be substantially greater than the vertical deformation.

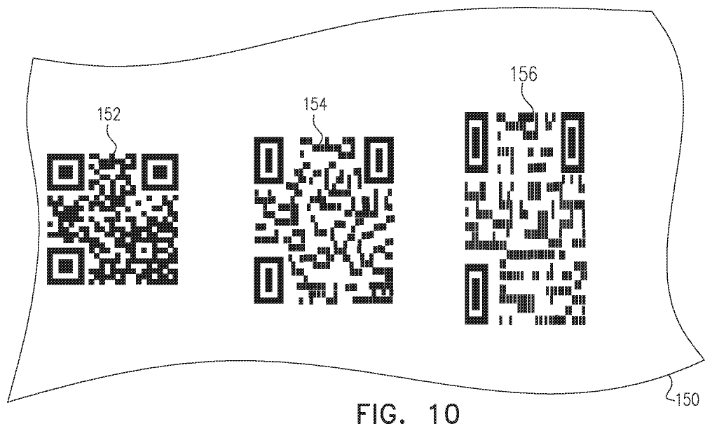

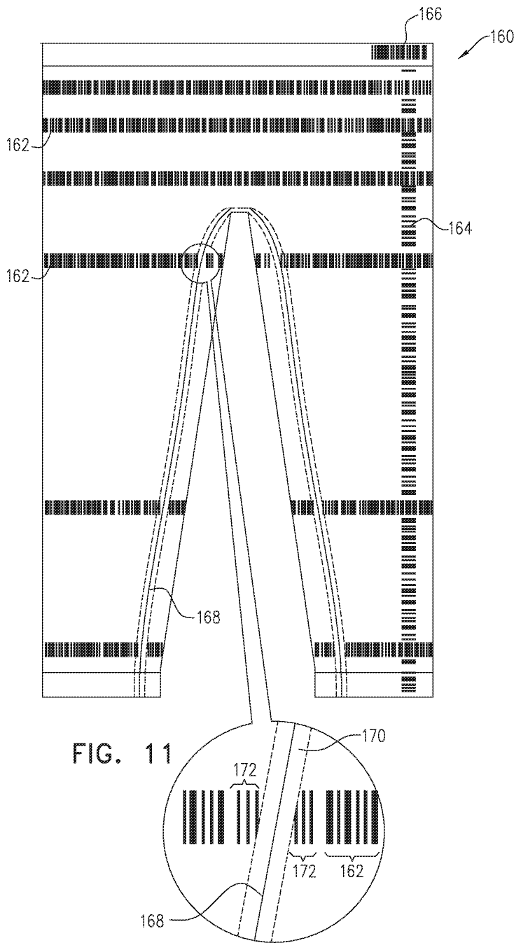

FIG. 10 is a schematic frontal view of graphical symbols 152, 154 and 156 in the pattern on a garment 150 that is used in measurement of body dimensions, in accordance with another embodiment of the invention. Garment 150 is shown as manufactured, i.e., unstretched, before a subject has put it on. To account for the horizontal deformation mentioned above, symbols 154 and 156 are elongated in a vertical direction, relative to the body of the subject. When a subject wears the garment, symbols 154 and 156 will expand horizontally, to shapes that are more nearly square. The processor will then be able more easily to recognize and decode these symbols, as well as measuring their deformation.

FIG. 11 is a schematic frontal view of a garment 160 used in measurement of body dimensions, in accordance with a further embodiment of the invention. Garment 160 comprises multiple horizontal measurement barcodes 162, as well as one or more vertical barcodes 164 and an identification code 166, as explained above.

In a typical manufacturing process, garment 160 is produced by cutting and then sewing a suitable patterned fabric to the appropriate shape and size. During sewing, for example using a flat stitch, two pieces of fabric are sewn together and the leftovers are cut off. Therefore, in an area 170 around a seam 168, different garments 160 of the same nominal style and size may vary, due to variations in the amount of fabric that was cut during sewing. These variations may affect the accuracy of measurement of body dimensions.

To remedy this problem, the pattern on garment 160 comprises a code 172, which is printed, knitted, or otherwise formed on the fabric in the area in which the fabric is to be cut and sewn. Code 172 will thus appear in proximity to seam 170 in the finished garment and will provide an indication of the extent of the fabric that is contained in the seam. In the pictured example, code 172 comprises reference lines (or other reference patterns), so that by counting the number of lines that remain visible after sewing both from the left and right of seam 168, the amount of fabric cut off during sewing can be estimated. The estimate can be made per garment 160 or even area by area along seam 168. The count and estimates may be carried out during production and/or by users, for example by capturing images along the seam using camera 30.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Measurement apparatus, comprising:
a garment comprising an elastic fabric having a predefined pattern extending across a surface thereof and configured to be worn over a part of a body of a subject, such that the elastic fabric stretches across the part of the body;
a camera, which is configured to capture images of the pattern while contacting and traversing across the surface of the fabric while the subject wears the garment; and
at least one processor, which is configured to process the images captured by the camera at multiple locations on the surface of the fabric so as measure a local deformation of the pattern at the multiple locations due to stretching of the fabric, and to compute a dimension of the part of the body responsively to the measured deformation.

2-12. (canceled)

13. The apparatus according to claim 1, wherein the camera comprises:
at least one image sensor;
a housing, having a front end configured to contact the fabric as the camera traverses across the surface of the fabric; and
objective optics mounted in the housing and configured to image a plane at the front end of the housing onto the image sensor.

14. The apparatus according to claim 13, wherein the image sensor is contained in a mobile computing device, and the housing comprises a fastener for attachment of the housing to the mobile computing device.

15. The apparatus according to claim 13, wherein the camera comprises a wireless interface for transmitting data with respect to the images from the camera to the at least one processor.

16. The apparatus according to claim 13, wherein the at least one image sensor comprises a plurality of image sensors configured to capture the images of the pattern along different, respective axes.

17. The apparatus according to claim 13, wherein the camera comprises a window mounted at the front end of the housing so as to contact the surface of the fabric.

18. The apparatus according to claim 17, wherein the window curves inward into the housing.

19. The apparatus according to claim 13, wherein the camera comprises one or more light sources disposed in the housing and configured to illuminate the fabric while the camera traverses across the surface.

20. The apparatus according to claim 19, wherein the one or more light sources comprise a plurality of light sources, which are positioned in the housing so as to illuminate the fabric from different, respective angles.

21. The apparatus according to claim 19, wherein the one or more light sources are configured to direct flash illumination toward the fabric while the camera traverses across the surface.

22. The apparatus according to claim 1, wherein the at least one processor is configured to output an indication of the computed dimension to a user of the apparatus.

23. The apparatus according to claim 22, wherein the predefined pattern comprises an identification code, which identifies the garment, and wherein the at least one processor is configured to process the images so as to read and validate the identification code, and to output the indication subject to finding the identification code to be valid.

24. The apparatus according to claim 22, wherein the at least one processor is configured to store values of the computed dimension, and to compare the computed dimension to the stored values, wherein the output is indicative of a change in the computed dimension relative to one or more of the stored values.

25. The apparatus according to claim 1, wherein the at least one processor is configured to identify, responsively to the computed dimensions, one or more articles of clothing of a size suitable to be worn by the subject.

26. The apparatus according to claim 1, wherein the at least one processor is configured to track the locations on the surface of the fabric at which the camera captures the images, and to prompt a user of the apparatus, responsively to the tracked locations, to shift the camera to an area of the garment in which the images of the pattern have not yet been captured.

27. A method for measurement, comprising:
providing a garment comprising an elastic fabric having a predefined pattern extending across a surface thereof and configured to be worn over a part of a body of a subject, such that the elastic fabric stretches across the part of the body;
while the subject is wearing the garment, capturing images of the pattern using a camera, while the camera contacts and traverses across the surface of the fabric;
processing the images captured by the camera at multiple locations on the surface of the fabric so as measure a local deformation of the pattern at the multiple locations due to stretching of the fabric; and
computing a dimension of the part of the body responsively to the measured deformation.

28-38. (canceled)

39. The method according to claim 27, wherein the camera comprises at least one image sensor and objective optics, which are mounted in a housing and configured to image a plane at a front end of the housing onto the image sensor, and wherein capturing the images comprises placing the front end of the housing in contact with the fabric as the camera traverses across the surface of the fabric.

40. The method according to claim 39, wherein the image sensor is contained in a mobile computing device, and wherein capturing the images comprises fastening the housing to the mobile computing device in alignment with the image sensor.

41-47. (canceled)

48. The method according to claim 27, and comprising outputting an indication of the computed dimension to a user.

49. The method according to claim 48, wherein the predefined pattern comprises an identification code, which identifies the garment, and wherein processing the images comprises reading and validating the identification code, wherein the indication is outputted subject to finding the identification code to be valid.

50. The method according to claim 48, and comprising storing values of the computed dimension, and comparing the computed dimension to the stored values, wherein outputting the indication comprises reporting a change in the computed dimension relative to one or more of the stored values.

51. The method according to claim 27, and comprising identifying, responsively to the computed dimensions, one or more articles of clothing of a size suitable to be worn by the subject.

52. The method according to claim 27, wherein processing the images comprises tracking the locations on the surface of the fabric at which the camera captures the images, and prompting a user, responsively to the tracked locations, to shift the camera to an area of the garment in which the images of the pattern have not yet been captured.

53. A garment, comprising an elastic fabric, which is configured to be worn over a part of a body of a subject, such that the elastic fabric stretches across the part of the body, the fabric having a predefined pattern, which extends across a surface of the fabric and comprises multiple graphical symbols, which are disposed at different, respective locations across the surface of the fabric and encode information identifying the respective locations.

54. The garment according to claim 53, wherein at least one of the graphical symbols encodes information identifying the garment.

55. The garment according to claim 53, wherein the graphical symbols comprise barcodes.

56. The garment according to claim 55, wherein the barcodes comprise two-dimensional matrix barcodes.

57. The garment according to any of claims 53-56, wherein the predefined pattern comprises one or more pattern colors that are distinguishable in the images from a background color of the fabric.

58. The garment according to claim 53, wherein the predefined pattern is formed in a pattern color that is indistinguishable to a human eye from a background color of the fabric.

59. The garment according to claim 53, wherein the predefined pattern is woven or knitted into the fabric.

60. The garment according to claim 59, wherein the woven or knitted pattern has a patterned texture that is distinguishable from a background texture of the fabric.

61. The garment according to claim 53, wherein the predefined pattern comprises a code, which is disposed in proximity to a seam in the garment and is indicative of an extent of the fabric that is contained in the seam.

62. The garment according to claim 53, wherein the predefined pattern comprises graphical symbols that are elongated in a vertical direction, relative to the body of the subject, while the elastic fabric is unstretched, and which expand horizontally when the subject wears the garment.

63-71. (canceled)
